SEQUENCE LISTING (7 sequences)

SEQ ID NO: 1 - protein, 34 aa, herpes simplex virus 1
NAATATRGRSAASRPTERPRAPARSASRPRRPVE

SEQ ID NO: 2 - protein, 15 aa, Human immunodeficiency virus
CGRKKRRQRRRPPQC

SEQ ID NO: 3 - protein, 9 aa, Human immunodeficiency virus
RKKRRQRRR

SEQ ID NO: 4 - protein, 16 aa, Drosophila Antennapedia
RQIKIWFQNRRMKWKK

SEQ ID NO: 5 - protein, 26 aa, Artificial
  Fusion peptide
GWTLNSAGYLLKINLKALAALAKKIL

SEQ ID NO: 6 - protein, 18 aa, Artificial
  Polypeptide
KLALKLALKALKAALKLA

SEQ ID NO: 7 - DNA, 73 bp, Artificial
  GATA4
ctcgagatctgccagcctgagataacagggccagcctgagataacagggccagcctgagataacaggaagctt

(19) United States
(12) Patent Application Publication
(10) Pub. No.: US 2010/0196338 A1
(43) Pub. Date: Aug. 5, 2010

(54) COMPOSITIONS AND METHODS FOR TREATING CARDIOVASCULAR DISEASE

(75) Inventors: Marc S. Penn, Beachwood, OH (US); Matthew Kiedrowski, Cleveland, OH (US)

Correspondence Address: TAROLLI, SUNDHEIM, COVELL & TUMMINO L.L.P., 1300 EAST NINTH STREET, SUITE 1700, CLEVELAND, OH, 44114, US

(73) Assignee: THE CLEVELAND CLINIC FOUNDATION, Cleveland, OH (US)

(21) Appl. No.: 12/522,714

(22) PCT Filed: Jan. 10, 2008
(86) PCT No.: PCT/US2008/050782

§ 371 Date: Apr. 1, 2010

Related U.S. Application Data
(60) Provisional application No. 60/879,736, filed on Jan. 10, 2007.

(51) Int. Cl. A61K 35/12; C12N 1/21; C12N 5/071; A61K 35/74; A61P 9/00

(52) U.S. Cl. 424/93.21; 435/252.3; 435/325; 424/93.2

(57) ABSTRACT

A fusion protein for treating cardiovascular disease includes a transcription factor (TF) and a cell-penetrating peptide (CPP). The fusion protein can be expressed from a cell that is delivered to the tissue being treated.

### RELATED APPLICATIONS

This application claims priority from U.S. Provisional Patent Application Ser. No. 60/879,736, filed Jan. 10, 2007, the subject matter of which is incorporated herein by reference.

### TECHNICAL FIELD

The present invention relates generally to compositions and methods for treating cardiovascular disease, and more particularly to proteins and cell-based methods for delivering proteins to treat cardiovascular disease.

### BACKGROUND OF THE INVENTION

Autologous cell transplantation has attracted significant attention for its potential applications in clinical medicine and for the treatment and prevention of congestive heart failure following myocardial infarction. Autologous cells have the advantage that they can be expanded and genetically engineered in vitro to express functional proteins for therapeutic application in the heart. While this approach offers great promise, it is limited by the fact that some proteins of interest are not easily secreted into the local tissue and/or internalized into surrounding cells.

### SUMMARY OF THE INVENTION

The present invention relates generally to compositions and methods for treating cardiovascular disease, and more particularly to compositions and methods for delivering non-secreted proteins to treat cardiovascular disease and/or promote cardiac remodeling, function and/or regeneration.

According to one aspect of the present invention, the composition can comprise a fusion protein that includes a non-secreted protein (e.g., transcription factor (TF)) and a cell-penetrating peptide (CPP). In one example, the fusion protein can comprise a GATA4 polypeptide and a VP22 polypeptide. The fusion protein can be provided by genetically engineering a cell to express the fusion protein. The cell expressing the TF-CPP fusion protein can be delivered to a subject to treat cardiovascular disease.

Another aspect of the invention relates to a cell genetically engineered to express a non-secreted protein-CPP fusion protein. The expressed fusion protein can include a TF-CPPP fusion, such as a GATA4-VP22 fusion protein.

A further aspect of the invention relates to a method for treating cardiovascular disease by preparing at least one cell delivery vehicle expressing a TF-CPP fusion protein. The at least one cell delivery vehicle is then delivered to a cardiac target site comprising at least one cardiac cell. In one example, the TF can comprise GATA4 and the CPP can comprise VP22.

Yet another aspect of the invention relates to a kit that comprises at least one cell delivery vehicle expressing a TF-CPP fusion protein.

### BRIEF DESCRIPTION OF THE DRAWINGS

The foregoing and other features of the present invention will become apparent to those skilled in the art to which the present invention relates upon reading the following description with reference to the accompanying drawings, in which:

FIGS. 1A-D show a GATA4:VP22 expression construct, a GATA4 binding sequence, and DNA binding activity of GATA4:VP22. FIG. 1A is a schematic representation of a plasmid construct of GATA4:VP22 made with a myc tag in the C-terminal end. FIG. 1B is a Western blot showing a transfected VP22/GATA4 plasmid construct expressed in HeLa cells and rat cardiac fibroblasts and probed with anti-myc monoclonal or anti-GATA4 polyclonal antibody. FIG. 1C is a schematic representation showing GATA4 promoter-luciferase constructs: GATA4 binding sequence containing 5′-(A/T)GATA(A/G)-3′ was inserted into pGL3-luciferase reporter between two restriction sites of XhoI and HindIII. FIG. 1D is a bar graph showing the results of a GATA4 promoter-luciferase construct transfected into HeLa cells. The cells are stably expressing GATA4 or GATA4:VP22. Luciferase result is shown as fold activation of luciferase activity in samples transfected with GATA4 promoter-luciferase constructs compared with the control transfected with the corresponding empty vector in three different corresponding cells: HeLa, HeLa GATA4, or HeLa GATA4:VP22. The experiments were performed in triplicates. The bar graph represents mean±S.E. *P<0.05 for comparing HeLa GATA4:VP22 group with the other two controls groups, HeLa GATA4 and HeLa, respectively;

FIGS. 2A-C are graphs showing the effect of cell-based intercellular delivery of GATA4:VP22 on left-ventricular (LV) contractility. Echocardiographic parameters of LV fractional shortening were performed exactly before cell transplantation in one-month myocardial infarcted rats as the baseline, and 4 and 6 weeks after cell transplantation. n≧6 in each group. FIG. 2A shows the percentage change from baseline of LV fractional shortening 4 weeks and 6 weeks after cell transplantation of RCF GATA4:VP22, RCF GFP:VP22, or RCF GFP measured by echocardiography. *p<0.05 for comparing GATA4:VP22 group with time-matched GFP:VP22 or GFP group. FIG. 2B shows wall thickening of anterior and inferior walls after cell transplantation of RCF GATA4:VP22, RCF GFP:VP22 or GFP. The bar graph represents mean±S.E. *p<0.01 for comparing GATA4:VP22 with GFP:VP22 or GFP group at 6 weeks following cell transplantation. FIG. 2C shows the change in the radial strains from baseline 6 weeks after cell transplantation of RCF GATA4:VP22, RCF GFP:VP22 or RCF GFP. While there was no change of the strain in the infarcted zone (anterior and lateral segments) in all three groups, strain improved in the border (posterior and anteroseptal segments) and the remote zones (inferior and septal segments) in the GATA4 treatment group, and it deteriorated in the two control groups. The bar graph represents mean±S.E. *p<0.05 for comparing GATA4:VP22 group with GFP:VP22 or GFP group in border and remote zones at 6 weeks following cell transplantation;

FIGS. 3A-B show changes in LV morphology as measured by decreasing collagen deposition in cell transplants of RCF expressing GATA4:VP22. FIG. 3A is an anatomic assessment after myocardial infarction in the rat. Representative Masson's trichrome-stained sections 6 weeks after cell transplantation of RCF expressing GATA4:VP22 (n=9) versus control groups of cell transplantation of RCF expressing GFP:VP22 (n=6), GFP (n=6) or PBS only injection (n=3). FIG. 3B shows collagen deposition (LV area percentage) analysis of infarcted rat hearts treated with RCF GATA4:VP22 and other control groups of RCF GFP:VP22, GFP and PBS. The bar graph represents mean±S.E. *p<0.05 for comparing GATA4:VP22 group with the other 3 control groups, respectively;

FIG. 4 is a graph showing the survival rate of infarct Lewis rats treated with RCF GATA4:VP22, GFP:VP22 and GFP;

FIGS. 5A-B show cardiac hypertrophy resulting from intercellular delivery of GATA4:VP22. FIG. 5A is a representative perpendicular cross section of rat hearts from 6 weeks GATA4:VP22 (n=9) treated and control groups of GFP:VP22 (n=6), GFP (n=6), or PBS (n=3) treated infarcted rats compared with normal rats. H&E staining analysis in 20× and corresponding 40× magnification of peri-infarcted myocardium. Scale bar 40 μm. FIG. 5B shows morphometric analysis of average cardiomyocyte cross-sectional diameter means (μm) from GATA4:VP22 treated and control groups of GFP:VP22, GFP, or PBS treated infarcted rats compared with normal rats. The bar graph represents mean±S.E. *p<0.05 for comparing GATA4:VP22 group with the other 4 control groups, respectively;

FIGS. 6A-F are immunofluorescent stains showing the induction of cardiac myosin over expression in vivo as a result of the intercellular delivery of GATA4:VP22. Immunofluorescence staining of myc conjugated with FITC and cardial myosin (CM) heavy chain conjugated with Alexor fluo 594 were performed on the tissue slides treated with RCF GATA4:VP22 (FIG. 6A and FIG. 6D), RCF GFP:VP22 (FIG. 6B and FIG. 6E) or RCF GFP groups (FIG. 6C and FIG. 6F). Pictures were taken in the peri-infarct zones around injected cells six weeks after cell transplantation with corresponding DAPI and myc merged colors (FIGS. 6A, 6B, and 6C) or DAPI, myc and CM merged colors (FIGS. 6D, 6E, and 6F). Scale bar 40 μm;

FIGS. 7A-H are BCL2 immunofluorescent stains showing the effect of intercellular delivery of GATA4 on cardiomyocyte apoptosis. BCL2 staining (FITC) in conjunction with antibody against cardiac myosin heavy chain in the infarcted rats treated with GATA4:VP22 (FIG. 7B and FIG. 7F) compared with the control GFP:VP22 (FIG. 7C and FIG. 7G) in the peri-infarct zone and other two controls either from healthy rats (FIG. 7A and FIG. 7E) or infarcted rats treated with GATA4:VP22 in remote areas (FIG. 7D and FIG. 7H). Pictures were taken with corresponding DAPI. Cardiac myosin and BCL2 merged colors (FIGS. 7A, 7B, 7C and 7D) or BCL2 only (FIGS. 7E, 7F, 7G and 7H). Scale bar 40 μm;

### DETAILED DESCRIPTION

The present invention relates generally to compositions and methods for treating cardiovascular disease, and more particularly to compositions and methods for delivering non-secreted proteins to treat cardiovascular disease and/or promote cardiac remodeling, function and/or regeneration. By non-secreted proteins it is meant proteins that are not normally secreted and/or internalized once secreted by cells or tissues being treated. The non-secreted proteins can comprise, for example, tissue factors (TF) that can be delivered to tissue, such as myocardial tissue.

The non-secreted proteins can be administered to the myocardial tissue of the subject by delivering (e.g., implanting, injecting, transplanting) to the myocardial tissue a cell-based delivery vehicle (e.g., genetically modified autologous cell) that expresses a fusion protein that includes a cell penetrating peptide (CPP) and the non-secreted protein (e.g., VP22-GATA4).

The present invention is based on the discovery that cell-based delivery of a fusion protein comprising a non-secreted protein, such as a transcription factor (TF), and a cell-penetrating peptide (CPP) can be used for treating ischemic cardiomyopathy. More particularly, the present invention is based on the discovery that a fusion protein including non-secreted TF GATA-box binding protein 4 (GATA4) and VP22 can be delivered to injured myocardium using cell therapy to promote ventricular remodeling and normal cardiac functioning. Based on this discovery, the present invention provides TF-CPP fusion proteins, cells genetically engineered to express TF-CPP fusion proteins, and methods for treating cardiovascular disease using such cells.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989, and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Conventional methods of gene transfer and gene therapy can also be adapted for use in the present invention. See, e.g., Gene Therapy: Principles and Applications, ed. T. Blackenstein, Springer Verlag, 1999, Gene Therapy Protocols (Methods in Molecular Medicine), ed. P. D. Robbins, Humana Press, 1997, and Retro-vectors for Human Gene Therapy, ed. C. P. Hodgson, Springer Verlag, 1996.

Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present invention, the term “allogeneic” refers to cells or tissues that are obtained from a donor of one species and then used in a recipient of the same species.

As used herein, the term “autologous” refers to cells or tissues that are obtained from a donor and then re-implanted into the same donor.

As used herein, the term “cardiovascular disease” refers to any structural or functional disorder, disease, or condition that affects the heart and/or blood vessels.

As used herein, the term “cell delivery vehicle” refers to a cell capable of expressing a TF-CPP fusion protein.

As used herein, the term “cell-penetrating peptide or CPP” refers to any polypeptide or polypeptides that facilitate delivery of a TF across a cell membrane.

As used herein, the term “fusion protein” refers to two distinct proteins, polypeptides, peptides, and/or fragments thereof not normally associated with each other in nature that are encoded by the same reading frame, resulting in two or more distinct proteins and/or fragments being “fused” together. The polynucleotide encoding the fusion protein may also contain in the same reading frame additional peptide or polypeptide sequences useful in the present invention, such as epitope-tag encoding sequences, affinity purification-tag encoding sequences, additional functional protein encoding sequences, and the like, or a combination of any two or more thereof.

As used herein, the term “GATA-box binding protein or GATA” refers to members of the GATA family of zinc finger TFs. Members of the GATA family include GATA1, GATA2, GATA3, GATA4, GATA5 and GATA6.

As used herein, the term “polynucleotide” refers to deoxyribonucleotides or ribonucleotides and polymers thereof in either single-stranded or double-stranded form. The term encompasses polynucleotides containing known nucleotide analogs, or modified backbone residues or linkages, which are synthetic, naturally occurring, and non-naturally occurring, which have similar binding properties as the reference polynucleotide, and which are metabolized in a manner similar to the reference polynucleotide. Examples of such analogs include, without limitation, phosphorothioates, phosphoramidates, methyl phosphonates, chiral-methyl phosphonates, 2-O-methyl ribonucleotides, and peptide-nucleic acids. Unless otherwise indicated, a particular polynucleotide also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The term polynucleotide may be used interchangeably with gene, cDNA, mRNA, oligonucleotide, and nucleic acid.

As used herein, the term “polypeptide” refers to a molecule composed of monomers (amino acids) linearly linked by amide bonds (also known as peptide bonds). The term indicates a molecular chain of amino acids and does not refer to a specific length of the product. Thus, peptides, dipeptides, tripeptides, oligopeptides, and proteins are included within the definition of polypeptide. This term is also intended to refer to the products of post-expression modifications of the polypeptide, for example, glycosylation, hyperglycosylation, acetylation, phosphorylation, and the like. A polypeptide may be derived from a natural biological source or produced by recombinant technology, but is not necessarily translated from a designated nucleic acid sequence. A polypeptide may be generated by any manner known in the art, including by chemical synthesis.

As used herein, the term “translocation” refers to transfer of a CPP and/or CPP fusion protein across a cell membrane such that the CPP and/or CPP fusion protein is internalized within the cell.

As used herein, the term “transcription factor” refers to a class of DNA-binding proteins that are able to directly bind DNA and facilitate initiation or repression of transcription.

One aspect of the present invention relates to a fusion protein comprising at least one TF and at least one CPP. As described in further detail below, the TF-CPP fusion protein may comprise a recombinant fusion protein, meaning that the fusion protein has been produced in a host cell that has been either transformed or transfected with a polynucleotide encoding the fusion protein or produces the fusion protein as a result of homolgous recombination. Other methods for producing the TF-CPP fusion protein, such as by chemical cross-linking, are also described in more detail below.

The TF can include any one or combination of known DNA-binding motifs capable of binding DNA and thereby facilitating initiation or repression of transcription. Generally, TFs may be categorized into well-known classes based on their DNA-binding motifs. Examples of DNA-binding motifs include, for example, helix-turn-helix (HTH), leucine zipper, helix-loop-helix (HLH), G-quadruplex, β-ribbon, and zinc finger motifs.

The TF may include at least one zinc finger motif. Two types of DNA-binding proteins have the zinc finger motif: the classic zinc finger proteins and the steroid receptors. The zinc finger motif takes its name from the loop of amino acids that protrudes from the zinc-binding site. Often described as Cys2/His2 fingers, zinc fingers are usually organized as a single series of tandem repeats. The stretch of zinc fingers may range from nine repeats that occupy almost the entire protein (e.g., as in TFIIIA), to providing just one small domain consisting of two fingers. The C-terminal portion of each finger forms α-helices that bind DNA, while the

N-terminal portion forms β-sheets. The non-conserved amino acids in the C-terminal portion of each zinc finger are responsible for recognizing specific DNA-binding sites.

In one example, the TF can include a GATA-box binding protein (GATA). Members of the GATA family include GATA1, GATA2, GATA3, GATA4, GATA5 and GATA6. The GATA TF can include any one or combination of GATA1, GATA2, GATA3, GATA4, GATA5 and/or GATA6 polypeptides having amino acid sequences substantially similar to native mammalian GATA1, GATA2, GATA3, GATA4, GATA5 and/or GATA6, respectively. The GATA polypeptide may also include any one or combination of GATA1, GATA2, GATA3, GATA4, GATA5 and/or GATA6 polypeptides encoded by a GATA1, GATA2, GATA3, GATA4, GATA5 and/or GATA6 polynucleotide, respectively. The GATA TF can also include GATA homologs, variants, and/or fragments thereof. Specific GATA1, GATA2, GATA3, GATA4, GATA5 and/or GATA6 polypeptide and polynucleotide sequences are known in the art and are available, for example, through publicly accessible databases such as the GenBank sequence database.

In an aspect of the invention, the TF may include at least one HTH motif. The HTH motif or HTH domain is a common denominator in basal and specific transcription factors. At its core, the HTH domain comprises an open tri-helical bundle, which typically binds DNA at its third α-helix. In structural terms, HTH domains comprise several elaborations on the basic 3-helical core, such as the tetra-helical bundle, the winged-helix, and the ribbon-helix-helix type configurations. HTH domains are also involved in a wide range of functions beyond transcription regulation, including, for example, DNA repair and replication, RNA metabolism, and protein-protein interactions. Examples of transcription factors having HTH domains, such as MBR-1, Oct-1 and the λ repressor are well known in the art.

The TF may also include at least one leucine zipper motif. The leucine zipper motif is a stretch of leucine residues that provides a dimerization motif and allows juxtaposition of the DNA-binding regions of each subunit. The leucine zipper motif forms an amphipathic helix in which the leucine residues of one protein protrude from the α-helix and interdigitate in parallel with the leucine residues of another protein, ultimately forming a coiled coil domain. The region adjacent to the leucine repeats is highly basic in each of the zipper proteins and thus comprises an ideal DNA-binding site. Examples of TFs having at least one leucine zipper motif are well known in the art and include, for example, CREB, fos and jun.

The TF may further include at least one HLH motif. The amphipathic HLH motif has been identified in some developmental regulators in addition to genes coding for eukaryotic DNA-binding proteins. Proteins with this motif have the ability to bind DNA and dimerize. Such proteins share a common type of sequence motif: a stretch of 40-50 amino acids containing two amphipathic α-helices separated by a linker region (the loop) of varying length. Most HLH proteins contain a region adjacent to the HLH motif itself that is highly basic and which is needed for binding to DNA. Members of this group are referred to as basic HLH or bHLH proteins. Examples of HLH proteins are well known in the art and include, for example, MyoD, Myf5, myogenin, and MRF4.

In another aspect, the CPP can include any protein, peptide, and/or polypeptide that facilitates delivery of a non-secreted protein, such as TF, across a cell membrane. Generally, CPPs of the present invention consist of positively-charged, short peptide sequences that have the ability to cross the plasma membrane into the cell interior in an energy- and receptor-independent manner (i.e., via endocytosis). It should be appreciated however, that CPPs which cross the plasma membrane in ways other than endocytosis are also included within the scope of the present invention.

By way of example, the CPP can include the VP22 tegument protein of herpes simplex virus type 1 (HSV-1). An important property of VP22 is that when applied to a surrounding medium, VP22 may be taken up by cells and accumulate in the nucleus of the cells. Fusion proteins including VP22 conjugated to GFP, thymidine kinase protein, and p53, for example, have been targeted to cells in this manner. The VP22 polypeptide can have an amino acid sequence substantially similar to native mammalian VP22 polypeptide. For example, a VP22 polypeptide can have the amino acid sequence NAATATRGRSAASRPTERPRAPARSASRPRRPVE (SEQ ID NO: 1). Other amino acid sequences corresponding to VP22 polypeptides, such as homologs, mutants, variants, and/or fragments thereof are known in the art and are available through the GenBank sequence database, for example.

Another example of a CPP that can be used in accordance with the present invention includes the human immunodeficiency virus (HIV) trans-activating protein (Tat). Tat is an 86-102 amino acid long protein involved in HIV replication. Tat can translocate through a plasma membrane and reach the cell nucleus, where it then transactivates the viral genome. One particular sequence of amino acids 48-60 (CGRKKRRQRRRPPQC) (SEQ ID NO: 2) from Tat is important for translocation, nuclear localization, and trans-activation of cellular genes. Numerous other Tat-derived short membrane translocation domains and sequences have been identified that possess translocation activity. Examples of such domains can include amino acids 37-72 and 49-58 (RKKRRQRRR) (SEQ ID NO: 3). Any of these fragments may be used alone or in combination with each other to enable translocation of the TF-CPP fusion protein into a cell.

Tat-derived polypeptides lacking the cysteine rich region (amino acids 22-36) and the carboxyl terminal domain (amino acids 73-86) have been also found to be particularly effective in translocation. Absence of the cysteine rich region and the carboxy terminal domain can prevent spurious trans-activation and disulfide aggregation. In addition, the reduced size of the transport polypeptide can minimize interference with the biological activity of the molecule being transported and increase uptake efficiency. Accordingly, use of CPPs comprising such Tat-derived polypeptides (i.e., those lacking the carboxyl terminal domain and/or the cysteine rich-region) may be used to improve the translocation efficiency of the TF-CPP fusion protein.

Still another example of a CPP that can be used in accordance with the present invention includes all or part of the Drosophila Antennapedia (Antp) homeodomain (HD) protein. The CPP may comprise the third helix of Antp-HD, which has cell penetration properties. The region responsible for translocation in Antp-HD has been localized to amino acids 43-58 (i.e., the third helix), a 16 amino acid long peptide rich in basic amino acids. The third helix has the amino acid sequence RQIKIWFQNRRMKWKK (SEQ ID NO: 4). This polypeptide has been used to direct biologically active substances to the cytoplasm and nucleus of cells in culture. Accordingly, the CPP of the present invention may comprise an Antp-HD polypeptide, an Antp-HD homolog, an Antp-HD variant, and/or an Antp-HD fragment, such as a fragment containing the third helix of Antp-HD.

In another aspect of the present invention, the CPP may include at least one signal sequence. Signal sequences of peptides are recognized by acceptor proteins that aid in mobilizing pre-proteins from the translation machinery to the membrane of appropriate intracellular organelles. Signal sequence-based translocators are thought to function by acting as a leader sequence (“leading edge”) to carry peptides and proteins into cells. The core hydrophobic region of a signal peptide sequence may be used as a carrier for cellular import of, for example, intracellular proteins. Synthetic membrane translocation domains and amino acid sequences containing such hydrophobic regions may also be able to translocate into cells. One particular hydrophobic region, known as the h region, consists of 7-16 non-conserved amino acids and has been identified in 126 signal peptides ranging in length from 18-21 amino acids. Accordingly, a CPP of the present invention may include any one or combination of these or other known signal sequences.

Membrane translocation sequences comprising signal sequence-based peptides coupled to nuclear localization sequences (NLSs) may also be used as CPPs. For example, the MPS peptide signal-sequence-based peptide I is a chimera of the hydrophobic terminal domain of the viral gp41 protein and the NLS from the 5V40 large antigen, and has been found to be active in membrane translocation. Additionally, the peptide signal-sequence-based peptide II is derived from the nuclear localization signal of NF-κB p50 and USF2. Any one or combination of known membrane translocation sequences, including those provided herein, may be used alone or in combination with the CPP of the present invention to deliver fused or conjugated TFs into a cell.

In another aspect of the present invention, the CPP may include transportan. Transportan is a fusion between the neuropeptide galanin and the wasp venom peptide mastoparan. It can be localized in both the cytoplasm and nucleus, and may comprise the amino acid sequence GWTLNSAGYLLKINLKALAALAKKIL (SEQ ID NO: 5). The mechanism of cell penetration by transportan is not clear; however, it is known to be energy-independent and that receptors and endocytosis are not involved. Accordingly, the CPP of the present invention can comprise transportan, transportan homologs, and/or fragments thereof. For example, a CPP comprising a transportan variant may include N-terminal deletions of about 1-6 amino acids as such deletions are known to increase translocational activity of transportan.

The CPP can also include an amphiphilic model peptide. Amphiphilic model peptide is a synthetic 18-mer (KLALKLALKALKAALKLA) (SEQ ID NO: 6). The only essential structural requirement for amphiphilic model peptides is a length of four complete helical turns. The membrane translocation sequence can cross the plasma membrane of various cell types, including mast and endothelial cells, for example, by both energy-dependent and energy-independent mechanisms. The translocation behavior of amphiphilic model peptide shows analogy to several membrane translocation domain sequences including, for example, Antp-HD and Tat. Accordingly, the CPP of the present invention can comprise any amphiphilic model peptide, homolog, variant, and/or fragment thereof.

While any of the CPPs (including domains and/or sequences and/or fragments thereof exhibiting membrane translocation activity) provided above may be used for the purpose of generating a TF-CPP fusion protein, it should be appreciated that other variations are also possible. For example, variations such as mutations (e.g., point mutations, deletions, insertions, etc.) of any of the sequences disclosed herein may be employed, provided that some membrane translocation activity is retained. Furthermore, it will be appreciated that homologs of CPPs from any other organism, including those of synthetic origin, may also be used.

The TF-CPP fusion protein may be prepared using techniques known in the art. In one method, a TF may be fused to a CPP using a suitable host, such as a eukaryotic or prokaryotic cell. For example, a cDNA encoding a TF-CPP fusion protein may be constructed to include nucleic acid sequences encoding both a TF and a CPP. The nucleic acid sequences may be in-frame and may be located downstream of an N-terminal leader sequence (e.g., a sequence comprising a 6-Histidine tag). The N-terminal leader sequence may enable purification of the expressed recombinant TF-CPP fusion protein using methods known in the art.

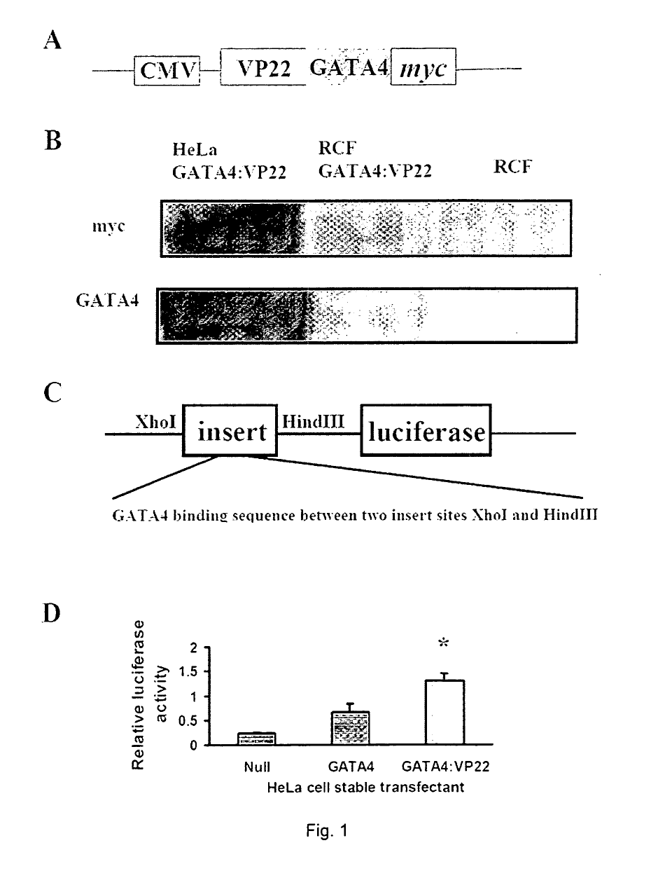

In one example, a recombinant GATA4-VP22 fusion protein may be prepared using a suitable eukaryotic host cell. As shown in FIG. 1A, for example, a GATA4-VP22 expression construct may be made by inserting a GATA4 polynucleotide in frame with the C-terminal portion of the plasmid pVP22/myc-His. The construct may also include a reporter gene, such as luciferase (FIG. 1C), to identify and/or measure effective transduction and/or transcription of a polynucleotide of interest. Using the FuGENE 6 system (Roche, Indianapolis, Ind.), for example, the plasmid may then be transfected into a line of expression-capable HeLa cells. Effective expression of the GATA4-VP22 fusion protein may then be monitored by, for example, the Luciferase Assay Kit (Promega, Madison, Wis.).

Other methods of forming fusion proteins can also be used. For example, a CPP can be chemically synthesized and then linked to a TF. Peptides can also be chemically cross-linked or coupled to larger peptides and proteins. The coupling may be permanent or transient, and may involve covalent or non-covalent interactions. Direct linkage, for example, may be achieved by localizing a functional group, such as a hydroxyl, carboxy or amino group, on the peptides. Indirect linkage can be achieved through a linking moiety, such as one or more of bi-functional cross-linking agents, for example. Coupling technologies are well known in the art.

Additionally or optionally, TF and CPP can be operably coupled with a linker sequence. Such a linker sequence may include a sequence of amino acids susceptible to cleavage by native enzymes (e.g., proteases), for example. The linker may comprise amino acid residues and/or hydrocarbon chains capable of connecting the TF and the CPP, for example, via peptide bonds. Useful linkers can also include natural and unnatural biopolymers. A non-exclusive example of a natural linker includes L-oligopeptides, while examples of unnatural linkers include D-oligopeptides, lipid oligomers, liposaccharide oligomers, peptide nucleic acid oligomers, polylactate, polyethylene glycol, cyclodextrin, polymethacrylate, gelatin, and oligourea.

Another aspect of the present invention relates to a cell genetically engineered to express a TF-CPP fusion protein. The cell may be autologous or allogeneic, and may comprise any kind of eukaryotic or prokaryotic cell capable of replicating a vector and/or expressing a heterologous gene encoded by a vector. Examples of cell-based expression systems are known in the art and include, for example, insect cell-based systems such as the TRIPLEXPRESS system, and mammalian expression systems such as CHO, HeLa and HEK 293 cell systems.

The cell that is genetically engineered to express the TF-CPP fusion protein can also be a cell that is biocompatible with tissue being treated (e.g., ischemic tissue, infarcted myocardial tissue). The biocompatible cells can include autologous cells that are harvested from the subject being treated and/or biocompatible allogeneic or syngeneic cells, such as autologous, allogeneic, or syngeneic stem cells (e.g., mesenchymal stem cells), progenitor cells (e.g., multipotent adult progenitor cells) and/or other cells that are further differentiated and are biocompatible with the tissue being treated. Where the cells genetically modified to express the TF-CPP fusion protein are transplanted into the tissue to be treated, the cell can be same cell type as the cells of the tissue being treated or a different cell type.

By way of example, where the tissue to be treated is infarcted myocardium the cells that are transplanted into the tissue to be treated can include cultured heart cells, skeletal myoblasts, fibroblasts, smooth muscle cells, and bone marrow derived cells. These cells can be harvested from the subject to be treated (i.e., autologous cells) and cultured prior to transplantation. Autologous cells are preferred to allogeneic and syngeneic cells in order to increase the biocompatibily of the cells upon transplantation and minimize the likelihood of rejection.

Methods for preparing genetically engineered cells are well known in the art and can include, for example, in vitro and ex vivo gene transfer techniques. Generally, ex vivo gene transfer refers to a process by which cells are removed from a subject, genetically manipulated (e.g., usually through in vitro transduction or transfection of polynucleotides into the cells), and then returned to the subject for therapeutic purposes. Similarly, in vitro gene transfer refers to a process by which cells are obtained (e.g., from a cell line) and then genetically manipulated.

In an example of the present invention, ex vivo gene transfer may be used to prepare a cell genetically engineered to express a GATA4-VP22 fusion protein. At least one host cell may first be obtained and cultured. The host cell can include, for example, a cardiac smooth muscle cell isolated from a biopsy taken from the myocardium of a subject. As used herein, the term “subject” refers to any mammalian subject including, for example, human beings, rats, mice, dogs, goats, sheep, horses, monkeys, apes, rabbits, cattle, etc. Methods for isolating cells from a biopsy or tissue sample are well known in the art and can include, for example, the use of digestive enzymes (e.g., collagenase) to deaggregate cells from the native cartilage. After isolating at least one smooth muscle cell, the cell may be placed into an appropriate in vitro culture system and propagated as needed.

A GATA4-VP22 expression construct may then be prepared. As used herein, the term “expression construct” refers to a polynucleotide having a transcribable region, i.e., a coding sequence that encodes a polypeptide of interest. The expression construct may enable expression of the polypeptide at a desired level (e.g., at a level that is constitutively higher than basal expression of the polypeptide of interest). As illustrated in FIG. 1A, the expression construct can include polynucleotides encoding GATA4 and VP22 polypeptides.

The GATA4-VP22 expression construct may additionally comprise at least one promoter. Generally, a promoter can include any polynucleotide sequence capable of binding RNA polymerase with increased affinity (e.g., as compared to a native promoter). As shown in FIG. 1A, the human cytomegalovirus (CMV) immediate-early enhancer/promoter may be included in the expression construct. The CMV promoter may provide a high level of expression of the GATA4-VP22 construct. Other examples of promoters are known in the art and include the bovine leukemia virus promoter, the SV40 promoter, and other cardiac tissue-specific promoters.

It should be understood that other polynucleotide sequences, such as enhancers that facilitate binding of DNA regulatory proteins may also be included in the expression construct. Additionally, it should be appreciated that other expression elements which facilitate detection and/or purification of the GATA4-VP22 fusion protein may also be included in the expression construct. As shown in FIG. 1A, the expression construct may include polynucleotides encoding the c-myc epitope to facilitate detection of the fusion protein (e.g., via anti-myc or anti-His antibodies). Additionally, the expression construct may include a polyhistadine (6×His) metal-binding tag to facilitate purification of the fusion protein (e.g., via affinity purification columns).

After an appropriate GATA4-VP22 expression construct has been prepared, the construct may be transfected into at least one cardiac smooth muscle cell via a delivery vector. The term “vector” as used herein refers to a replicatable nucleic acid expression system, such as an autonomous self-replicating circular DNA or virus, which is able to transfer an expression construct from one host to another. Viral vectors can include, for example, adenoviruses and lentiviruses. Non-viral vectors can include, for example, DNA plasmids and microscopic containment vehicles. Examples of microscopic containment vehicles can include liposomes, nanocapsules, nanoparticles, micelles, synthetic phospholipids, gas-dispersions, emulsions, microemulsions, nanospheres, and the like. As described in Example 1, for instance, a suitable vector for delivery of the GATA4-VP22 expression construct to the cardiac smooth muscle cell may include the pVP22/GATA4 plasmid.

Successful transfection of the vector into the host cell and transcription of the expression construct may be measured using any one or combination of known detection methods. For example, a detectable marker, such as green fluorescent protein (GFP) or luciferase may be included in the vector. For instance, the vector pGL3 includes the luciferase marker. Thus, when pGL3 has been effectively transfected into a cell and then transcribed, the luciferase marker may be expressed and then assayed to verify effective transfection and/or transcription.

The present invention also relates to a method of treating cardiovascular disease. “Cardiovascular disease” according to the present invention can include any structural and/or functional disorders, diseases, and/or conditions that affect the heart and/or blood vessels. Examples of cardiovascular disease can include, but are not limited to, arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.

In one step of the method, a cardiac target site is identified in a subject having a cardiovascular disease. As used herein, the term “cardiac target site” refers to an anatomical site or structure associated with a particular cardiovascular disease. The cardiac target site may further comprise at least one cardiac cell including, for example, cardiac progenitor cells, cardiac muscle cells, cardiac smooth muscle cells, cardiomyocytes, cardiac epithelial cells, cardiac endothelial cells, fibroblasts, cardiofibroblasts, cardiac electro-conducting cells, and combinations thereof. For example, where a subject has suffered a myocardial infarction, a portion of the left ventricular myocardium may have been damaged. Thus, the damaged portion of the left ventricular myocardium may comprise the cardiac target site, and a damaged cardiac smooth muscle cell may comprise the at least one cardiac cell.

Various methods known in the art may be used to identify the cardiac target site. For example, methods such as contrast-enhanced MRI, CT, PET, electrocardiogram, fluoroscopy, echocardiography, and/or histological analysis may be used to identify the cardiac target site. As described in Example 1, for instance, echocardiography may be used to detect various anatomical parameters indicative of myocardial damage following left ventricular ischemia. For example, parameters such as shortening fraction and anterior/inferior left ventricular wall thickening may be derived from the echocardiogram. These parameters may then be compared to control parameters, such as shortening fraction and wall thickness values derived from a non-diseased subject, for example, to identify the cardiac target site.

After the cardiac target site has been identified, at least one cell delivery vehicle expressing a TF-CPP fusion protein may be prepared as described above. For instance, the at least one cell delivery vehicle can comprise any type of cell, such as a eukaryotic or prokaryotic cell, and may be autologous or allogeneic. The at least one cell delivery vehicle can be derived from the tissue of a subject, such as from the myocardium, and may be genetically engineered using, for example, ex vivo and/or in vitro gene transfer techniques. In one particular example of the method, at least one cardiac smooth muscle cell may be isolated from the myocardium of a subject and then, transfected with a GATA4-VP22 expression construct.

The at least one cell delivery vehicle may then be delivered to the cardiac target site using known administration routes and techniques. For example, the at least one cell delivery vehicle can be administered by parenteral, subcutaneous, intravenous, intraarticular, intraarterial, intrathecal, intramuscular, intraperitoneal, or intradermal injections, or by transdermal, buccal, oromucosal, or ocular routes. Administration may be achieved using an appropriate delivery device, such as a needle, cannula, catheter, or the like. The appropriate route may be selected depending on the nature of the at least one cell delivery vehicle used, the cardiovascular disease to be treated, and an evaluation of the age, weight, sex and general health of the subject.

Suitable doses of the at least one cell delivery vehicle may be readily determined by one of skill in the art, depending upon the cardiovascular disease being treated, as well as the health, age and weight of the subject, for example. In general, selection of an appropriate “effective amount” or dosage of the at least one cell delivery vehicle may also be based upon the type of cell used and the identity of the TF-CPP fusion protein. The method and route of administration may also affect the dosage and amount of the at least one cell delivery vehicle delivered to the cardiac target site. Further, the amount of the at least one cell delivery vehicle required to produce a suitable response in a subject without significant adverse side effects may vary depending upon these factors. Suitable doses may be readily determined by persons skilled in the art.

Where a subject has suffered ischemic damage to the left ventricular myocardium, for example, the at least one cell delivery vehicle may be directly injected into the subject's left ventricle via a port on the heart wall. Alternatively, the at least one cell delivery vehicle may be injected into a pulmonary vein so that the cell delivery vehicle may travel through the left atrium and then into the left ventricle. Delivery of the at least one cell delivery vehicle to the cardiac target site may be monitored using any one or combination of known imaging techniques, such as those listed above.

Upon delivery of the at least one cell delivery vehicle to the cardiac target site, the at least one cell delivery vehicle may express the GATA4-VP22 fusion protein. The GATA4-VP22 fusion protein may then be secreted from the at least one cell delivery vehicle so that a portion of the fusion protein contacts a portion of at least one cardiac cell. For example, the VP22 portion of the fusion protein may contact a portion of the plasma membrane of a cardiac smooth muscle cell at the cardiac target site. The VP22 portion of the fusion protein, along with the GATA4 portion, may then be translocated across the plasma membrane of the at least one cardiac smooth muscle cell via an energy- and receptor-independent mechanism.

Translocation of the fusion protein allows the GATA4 polypeptide to then cross the nuclear membrane of the at least one cardiac smooth muscle cell and bind to its respective binding site on the DNA of the cardiac smooth muscle cell. Binding of GATA4 may then induce the expression of BCL2, an anti-apoptotic protein, which may prevent or reduce apoptosis in the smooth muscle cell. Reduction or prevention of apoptosis in the at least one cardiac smooth muscle cell may mitigate apoptosis, promote restoration of smooth muscle cell function, and facilitate cardiac remodeling at the cardiac target site.

The present invention further relates to a kit designed for delivery of at least one cell delivery vehicle expressing a TF-CPP fusion protein. The kit may be used in a clinical setting, in a research setting, or both, and may take a variety of forms. For example, the kit may include a pharmaceutical composition that comprises at least one cell delivery vehicle capable of expressing a TF-CPP fusion protein. Alternatively, the kit may include the components needed to prepare at least one pharmaceutical composition for cell delivery vehicle capable of expressing a TF-CPP fusion protein. For example, the kit may include at least one host cell, a TF-CPP expression construct, a suitable vector, appropriate buffers and culture media, and directions for use.

Another form of the kit may include a pharmaceutical composition comprising at least one cell delivery vehicle capable of expressing a TF-CPP fusion protein. The pharmaceutical composition may additionally include a physiologically acceptable diluent or a pharmaceutically acceptable carrier, such as sterile water or sterile isotonic saline. As used herein, the term “pharmaceutically acceptable carrier” refers to any and all solvents, dispersion media, coatings, antibacterial and antifungal agents, isotonic and absorption delaying agents, and the like, compatible with administration of the formulation to a subject. The appropriate carrier will be evident to those skilled in the art and will depend in part upon the route of administration.

Additional components that may be present with in the pharmaceutical composition include adjuvants, preservatives, chemical stabilizers, and/or other proteins. Typically, stabilizers, adjuvants, and preservatives are optimized to determine the best formulation for efficacy in a subject. Exemplary preservatives include, but are not limited to, chiorobutanol, potassium sorbate, sorbic acid, sulfur dioxide, propyl gallate, the parabens, ethyl vanillin, glycerin, phenol, and parachiorophenol. Suitable stabilizing ingredients include, for example, casamino acids, sucrose, gelatin, phenol red, N-Z amine, monopotassium diphosphate, lactose, lactalbumin hydrolysate, and dried milk.

Other components of the pharmaceutical composition can include, for example, surface active substances (e.g., hexadecylamine, octadecylarnine, octadecyl amino acid esters, lysolecithin, dimethyl-dioctadecylammonium bromide), methoxyhexadecyiglycerol, pluronic polyols, polyamines (e.g., pyran, dextransulfate, poly IC, carbopol), peptides (e.g., muramyl dipeptide, dimethyiglycine, tuftsin), oil emulsions, mineral gels (e.g., aluminum phosphate), immune stimulating complexes, liposomes, polysaccharides, lipopolysaccharides, and/or other polymers.

The following example is for the purpose of illustration only and is not intended to limit the scope of the claims, which are appended hereto.

### Example 1

### Introduction

GATA4 is critical for the viability and hypertrophic response of cardiac myocytes following myocardial pressure overload. Consistent with these findings, GATA4 has also been recently shown to directly induce expression of BCL2 in cardiac myocytes. These findings would suggest that the local over-expression of GATA4 could lead to local cardiac hypertrophy and improved cardiac myocytes survival. In this study, we wanted to determine the effects of local GATA4 over-expression in a model of ischemic cardiomyopathy. However, local delivery of GATA4 is problematic because transcription factors are not normally secreted nor internalized by surrounding cells.

Cell-based gene therapy has been shown to be an effective strategy for stimulating angiogenesis and improving heart function. Moreover, cell-penetrating peptides (CPPs) as vectors for the transmembrane intercellular delivery of fused proteins have emerged as a significant tool to modulate biological activities. Therefore, to allow for sustained local transcription factor delivery, we engineered a cell-based gene therapy strategy to establish sustained intercellular protein delivery using a chimeric protein encoding GATA4 and the CPP VP22. This strategy has been shown to allow for the delivery of non-secreted protein into myocardial tissue via autologous cells transplantation. Although a number of studies have examined the ability of transducible protein VP22 to deliver functional proteins to inhibit tumor growth both in vitro and in vivo, little is known about its potential applications in combination with autologous cell transplantation for the delivery of proteins to myocardial tissue.

In the present study we quantify the effects of sustained GATA4:VP22 release into the infarct borderzone in a rat model of ischemic cardiomyopathy induced by LAD ligation 1 month prior to cell delivery. We aim to show that the cell-based sustained delivery of non-secreted functional proteins offers a potential novel strategy for the study of non-secreted proteins as well as a potential treatment option for the optimization of cardiac function of the injured myocardial tissue.

### Materials and Methods

Plasmid Constructs

GATA4:VP22 constructs were made by inserting GATA4 construct (kindly provided by Dr. J. D. Molkentin at Cincinnati Children's Hospital Medical Center) into C-terminals of VP22 in frame in the plasmid pVP22/myc-His (Invitrogen, Carlsbad, Calif., USA). The CMV promoter drove GATA4 expression in the vector pVP22/GATA4. The anticipated molecular weight for fusion protein GATA4:VP22 was about 80 kDa. Plasmids of pVP22/GFP and GFP were constructed and transfected into rat cardiac fibroblasts.

Luciferase GATA4 promoter reporter construct containing GATA4 binding site was made using the GATA motif (A/T)GATA(A/G). The insert containing (A/T)GATA (A/G) motif with 3 repeats was pGL3 vector (Promega Corp., Madison, Wis., USA) at the XhoI and HindIII sites. The insert of DNA sequences from 5′ to 3′ are pGL3 new GATA insert was as following (FIG. 1C): (XhoI) site CTCGAGATCTGCCAGCCTGAGATAACAGGGCCAGCCTGAGATAACAGGGCCAGC CTGAGATAACAGGAAGCTT (HindIII) (SEQ ID NO: 7).

Transfection and Luciferase Activity

Plasmids used for transfection studies were prepared using FuGENE 6 (Roche, Indianapolis, Ind., USA) as described previously.

The insert containing DNA sequence (A/T) GATA (A/T) motif in pGL3 luciferase reporter as described above was transfected into HeLa, HeLa stably expressing the GATA4 expression construct and HeLa stably expressing the GATA4:VP22 construct. Empty pGL3 vector containing no insert was transfected into the three corresponding cell lines was used as a negative control. In these experiments promoter activity was determined by the ratio of luciferase activity from GATA4 binding sequence promoter driven luciferase activity to empty vector for each of the cell lines studied. Cells were transfected overnight prior to assaying for luciferase activity using the Luciferase Assay Kit (Promega), which quantifies firefly luciferase.

Cell Culture and Western Blot Analysis

Rat cardiac fibroblasts were harvested from rat heart and cultured in vitro and plasmids used for transfection were prepared using FuGENE6 (Roche, Indianapolis, Ind., USA) and stably transfected into rat cardiofibroblasts. Western blot analysis was performed as described previously, probed with anti-myc monoclonal antibody (Santa Cruz Co. CA, USA), and anti-GATA4 polyclonal antibody (Santa Cruz).

Myocardial Infarction and Cell Delivery

The Animal Research Committee approved all animal protocols, and all animals were housed in the Association for the Assessment and Accreditation of Laboratory Animal Care animal facility of the Cleveland Clinic Foundation. Myocardial infarction was induced by ligation of the left anterior descending coronary artery in male Lewis rats weighing 250-300 gram. The presence of infarct was assessed by echocardiography immediately after chest closure. Infarcted animals were randomized into three treatment groups: RCF-GFP:VP22, RCF-GFP and RCF-GATA4:VP22.

Four weeks after infarction, the rat hearts were exposed by thoracotomy as described. 150 μl PBS without cells (sham) or RCF-GFP:VP22, GATA4:VP22 and GFP (1×106 cells) were injected through a 30½ G gauge needle (PrecisionGlide Needle, BD Bioscience, Franklin Lakes, N.J.) around the border zone of the left ventricular (LV) infarcted areas in 5 divided doses.

Echocardiography

We performed echocardiography immediately before and 4 and 6 weeks after cell transplantation. Echocardiography was performed in conscious rats sedated by ketamine (80 mg/kg body weight, i.p.) by using GE Vivid 7 machine (GE Medical, Milwaukee, Wis., USA) equipped with a 14-MHZ linear transducer. M-mode and 2-dimensional cross-sectional echocardiography data were acquired at a papillary muscle level and digitally stored for further analysis.

From M-mode tracings, anatomical parameters in diastole and systole were obtained. Shortening fraction (SF) was derived from LV cross-sectional area in 2D short axis view: SF=[(LVEDD-LVESD)/LVEDD]×100 where LVEDD and LVESD correspond to LV end-diastolic diameter and end-systolic diameter.

The wall thickening (WT) for anterior and inferior wall was derived from the view in which M-mode cursor was positioned perpendicular to the LV anterior and inferior walls. The wall thickening was calculated as WT %=(WTHs−WTHd)/WTHd×100%, where WTH stands for the actual thickness of the ventricular wall (mm), while d and s stand for end diastole and end systole.

To assess radial strains, we analyzed 2-dimensional high-frame rate (frame rate range 92-123 fps) echocardiography data using Echopac PC workstation (GE Medical, Milwaukee, Ws). This recently validated software tracks the speckle inherent to a standard two dimensional echocardiography image to obtain radial strain in six LV segments. Finally, data from at least two cardiac cycles were then averaged.

Histological Analysis and Immunohistochemistry

Rats were euthanized (KCl2 mEq IV to arrest the heart in diastole) under deep anesthesia. The hearts were excised and with histochoice. The hearts were cut into 3 transverse slices. The middle slice was embedded in paraffin and processed for hematoxylin-eosine and Masson's trichrome staining and the others were embedded in paraffin and used for immunofluorescence. Collagen deposition was evaluated from sections (5 μm thickness) of the paraffin-embedded tissue stained with Masson Trichrome stain as described previously by observers blinded to the identity of individual samples. Images of tissue sections were acquired using an ArtixScan 4000tf (Microtek, Carson, Calif., USA) slide scanner with PathScan Enabler (Meyer Instruments, Houston, Tex.) at a resolution of 4000 dpi. Software ImagePro Plus (Media Cybernetics, Silver Spring, Md., USA) was used to quantify percentage of collagen deposition. Percentage length of collagen deposition (%) was expressed as percentage circumference occupied by collagen deposition wall of total LV circumference; percentage area of collagen deposition (%) is expressed as percentage area occupied by collagen deposition area of total LV area.

Myocyte cross-sectional dimensions in the peri-infarct zone were measured from hematoxylin and eosin-stained slides. Images were obtained by a blinded observer using Leica DMR upright microscope (Leica Co., Heidelberg, Germany) with cooled CCD camera (Q-imaging Retiga Ex, British Columbia, Canada). Morphometric analysis of H&E-stained tissues was performed using ImagePro Plus software at magnification of 200×. Cross-sectional diameters of five randomly chosen cardiac myocytes were measured as described in each of eight randomly chosen fields per animal by two observers blinded to the identity of the animal from which the images were obtained.

In order to trace the injected cells in the peri-infarct zone, immunofluorescence was performed on histochoice-fixed 5-μm-thick sections with antigen retrieval followed by incubation with the specified antibodies as described. Since the VP22 chimeric proteins have myc tags in the C-terminals, the tissue sections were first incubated with goat polyclonal anti-myc with FITC (Abcam Inc., Cambridge, Mass.) and then incubated with monoclonal cardiac myosin heavy chain (Chemicon Internation Corp.) with secondary donkey anti-mouse alexor 594 (Invitrogen). In order to testify BCL2 expression induced by intercellular delivery of GATA4, tissue sections were double stained with antibody of BCL2 (Santa Cruz) with FITC as secondary antibody in conjunction with cardiac myosin heavy chain with alexor fluor 594 as secondary antibody (Invitrogen). Eight digital images were obtained by Leica TCS SP2 AOBS confocal laser scanning system (Leica Co., Wetzlar, Germany) using ultraviolet (351/364 nm), 488 nm argon and krypton/argon (568 nm).

Statistical Analysis

All data were presented as mean±SE. Comparisons between groups for continuous variables were made by Student t test. For segmental radial strain data, we used repeated measures analysis of variance to analyze the change of segmental radial strains during a 6-week treatment period, followed by analysis of contrasts. Results were considered statistically significant if p<0.05.

### Results

GATA4:VP22 can Make Intercellular Delivery and Bind into Nucleus

A plasmid GATA4:VP22 was constructed with myc tag in the C-terminal of the fusion proteins (FIG. 1A). We transfected this construct into HeLa cells and rat cardiac fibroblasts (RCF). Western blot using antibodies against myc or GATA4 epitopes showed that the chimeric protein was expressed with the predicted molecular weight in both cell types although expression in RCF was less than in HeLa (FIG. 1B). No bands were observed in wild type RCFs (FIG. 1B). In order to verify that the chimeric GATA4 protein retained its activity as a transcription factor that activates a GATA4 responsive promoter we used a luciferase reporter assay for GATA4 responsive promoters in HeLa cells. GATA4 binding sequence containing 5′-(A/T)GATA(A/G)-3′ was inserted into the pGL3-luciferase construct (FIG. 1C). The reporter construct was transiently transfected into wild type HeLa cells, HeLa cells stably expressing empty vector, GATA4 or GATA4:VP22. The results of luciferase assay demonstrate that GATA4:VP22 chimeric protein maintained the ability to activate GATA4 responsive promoters (FIG. 1D).

Intercellular Delivery of GATA4 Improved LV Function of Infarcted Heart

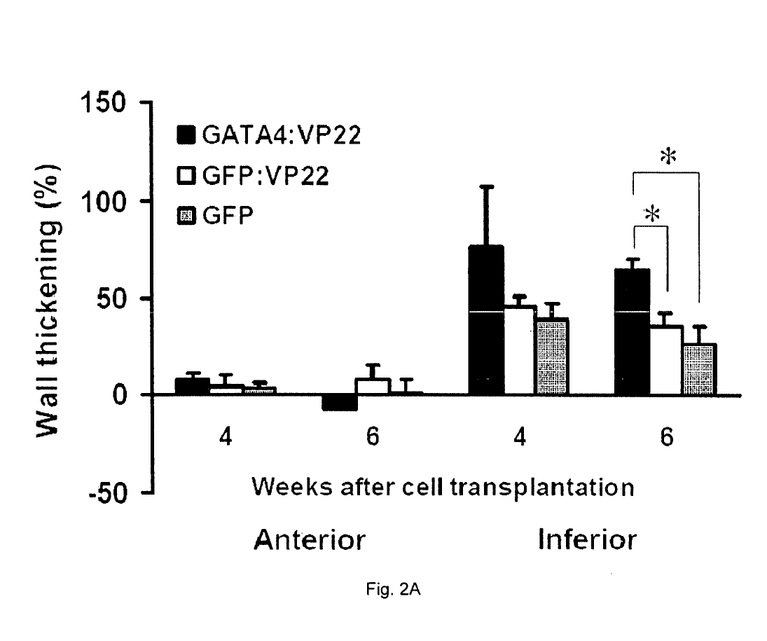

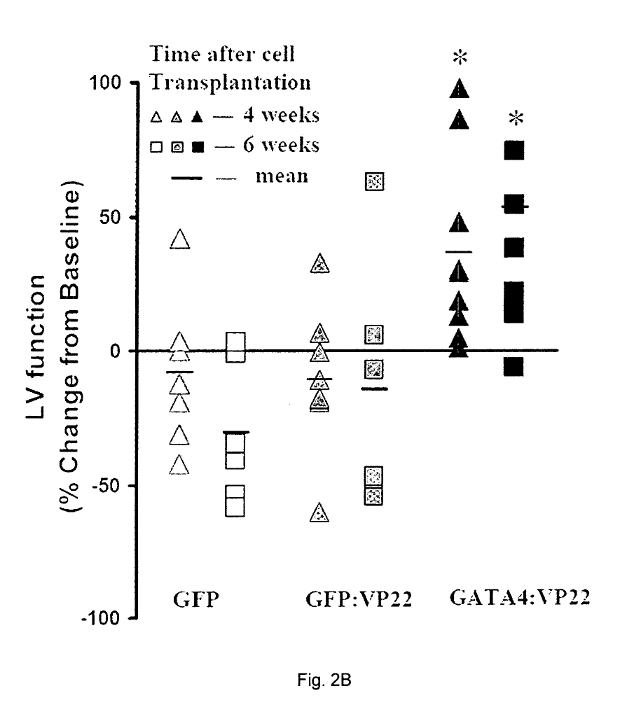

We investigated the effects of intercellular delivery of GATA4 on LV function after myocardial infarction. LV function and contractility were evaluated by echocardiography. Rat cardiac fibroblasts (RCFs) stably expressing GATA4:VP22 or control groups of RCF stably expressing GFP:VP22 or GFP were directly injected into the peri-infarct zones of Lewis rats one-month after LAD ligation in 5 divided doses around the infarct zone as described. Baseline echocardiography was performed within 2 days before cell transplantation (4 weeks after LAD ligation) and 4 and 6 weeks after cell transplantation (Total 10 weeks after LAD ligation). Four weeks after cell transplantation, the percentage change in shortening fraction of left ventricle from baseline in GATA4:VP22 (37% increase) group was significantly increased compared to GFP:VP22 (−11%, p=0.006) or GFP (−9%, p=0.02) control (FIG. 2A). Six weeks after cell transplantation the improvement in fractional shortening was maintained in hearts treated with GATA4:VP22 (54% increase) compared with GFP:VP22 (−15%, p=0.02) and GFP (−31%, p=0.003) control groups (FIG. 2A).

Systolic and diastolic thickness of the anterior and inferior walls is presented in the following Table. The wall thickening of inferior wall was significantly increased six weeks after cell transplantation with GATA4:VP22 (65%) compared with control groups (GFP:VP22, 35%, p=0.01; GFP 26%, p=0.001, FIG. 2B). In contrast, there was no significant difference in anterior wall thickening compared with controls (FIG. 2B).

TABLE

| | Anterior | Anterior | Anterior | Anterior | Inferior | Inferior | Inferior | Inferior |
|---|---|---|---|---|---|---|---|---|
| | diastolic | systolic | Diastolic | Systolic | diastolic | Systolic | Diastolic | Systolic |
| | 4 w(mm) | 4 w(mm) | 6 w(mm) | 6 w(mm) | 4 w(mm) | 4 w(mm) | 6 w(mm) | 6 w(mm) |
| GATA4:VP22 | 1.1 | 1.2 | 0.9 | 0.8 | 1.4 | 2.2 | 1.5 | 2.5 |
| GFP:VP22 | 1.1 | 1.1 | 1.2 | 1.2 | 1.9 | 2.7 | 2.5 | 3.3 |
| GFP | 1.2 | 1.2 | 1.2 | 1.2 | 1.8 | 2.5 | 2.0 | 2.5 |

We observed a significant difference in segmental radial LV strains in our baseline measurements four weeks after myocardial infarction. The radial strains were lowest in the infarct zone (anterior and lateral segments), higher in the border (posterior and anteroseptal segments) and increased in the zones remote from myocardial infarction (inferior and septal segments) (p<0.001; see FIGS. 9A-B). After six weeks of treatment, there were no differences between groups in the infarct zone (p=NS); however radial strains improved significantly more in the non-infarcted zones in GATA4 treatment group compared with GFP:VP22 group (p=0.045) and GFP group (p=0.031) (FIG. 2C).

Cell-Based Intercellular Delivery of GATA4 Result Inremodeling of the Infarcted Hearts and Increased the Survival Rate of Infarcted Rats

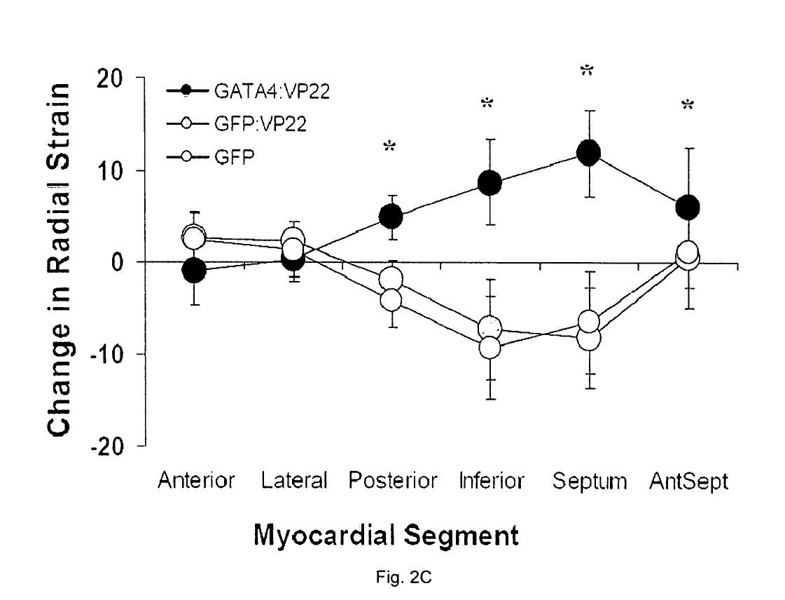

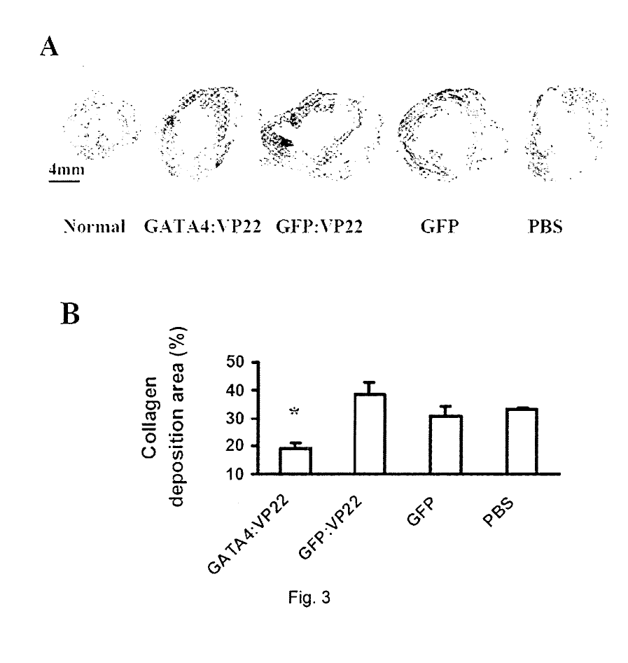

Myocardial fibrosis: To further investigate if the intercellular delivery of GATA4 influenced the morphology of the heart, Masson's trichrome staining was performed to quantify the extent of left ventricular fibrosis. Anatomic assessments of Masson's trichrome staining are shown in FIG. 3A. The percent area of collagen deposition in the left ventricle was significantly reduced 6 weeks after cell transplantation of GATA4:VP22 expressing RCF (19%) compared with GFP:VP22 (39%, p=0.0004), GFP (31%, p=0.007) and PBS (33%, p=0.003) and the percent of the LV circumference in which there was collagen deposition was also reduced significantly in the GATA4:VP22 compared to GFP:VP22, GFP and PBS (data not shown).

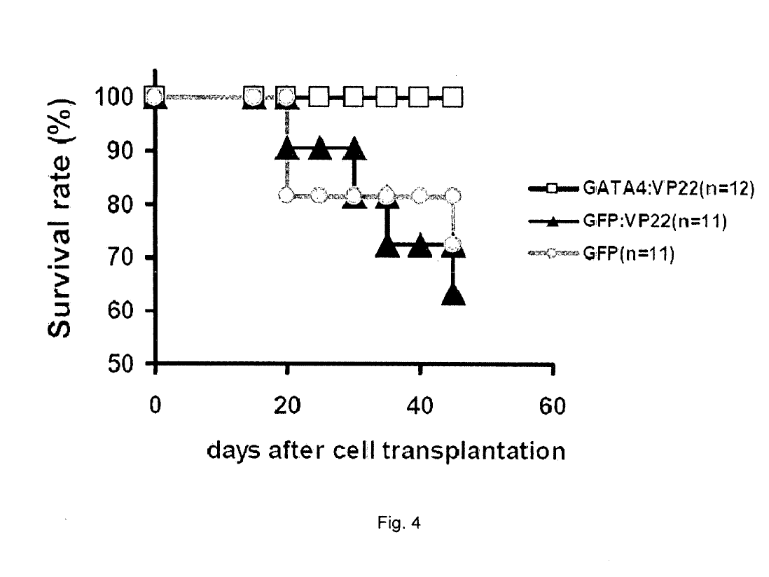

To evaluate the safety of using CPP VP22 to deliver GATA4 in infarcted rat hearts, the survival rates of each group were analyzed during 6 weeks after cell transplantation. FIG. 4 showed that only the groups of infarcted rats treated with GATA4:VP22 has 100% of survival rate under our experimental conditions. Also, the survival rates of the GATA4:VP22 treated rats (FIG. 4) were clearly higher than those in other corresponding controls within 6 weeks of follow-up.

These data demonstrated that, in addition to improving the LV function, cell transplantation of RCF stably expressing GATA4:VP22 could also improve the morphology of infarcted hearts by reducing fibrosis of infarcted myocardium. More significantly, the analysis of survival rates indicated that this strategy is well tolerated and could improve the survival rate of infarcted rats, which suggested that cell-based sustained intercellular delivery of GATA4 might have prevented negative remodeling late after myocardial infarction.

Cardiac Myocyte Hypertrophy: GATA4 could induce cardiac hypertrophy and can temporarily protect heart by reduction of wall stress and the subsequent sparing of energy and oxygen consumption, thus compensating for conditions such as myocardial infarction. Therefore we examined the extent of cardiac hypertrophy by measuring myocyte cross-sectional areas in the infarct border zone and remote from the border zone 6 weeks following cell transplantation with GATA4:VP22 expressing RCF or control groups.

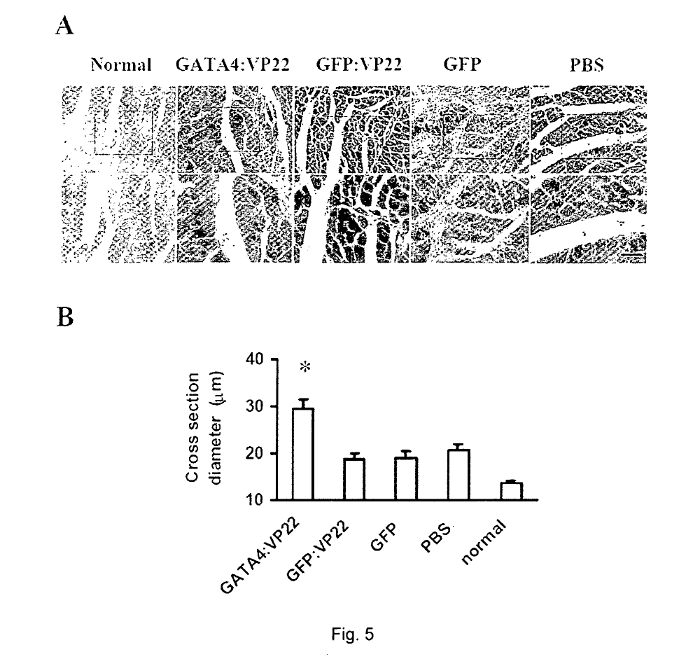

We observed cardiac myocytes hypertrophy in those animals that received GATA4:VP22 expressing RCF (FIG. 5A). Microscopic analysis of H&E-stained sections revealed that the diameter of cross-section of myocardium in the peri-infarct zone with treatment of GATA4:VP22 (30 μm) was significantly increased compared with all other control groups (GFP:VP22: 19 μm, p=0.0007; GFP 19 μm, p=0.001) (FIG. 5B).

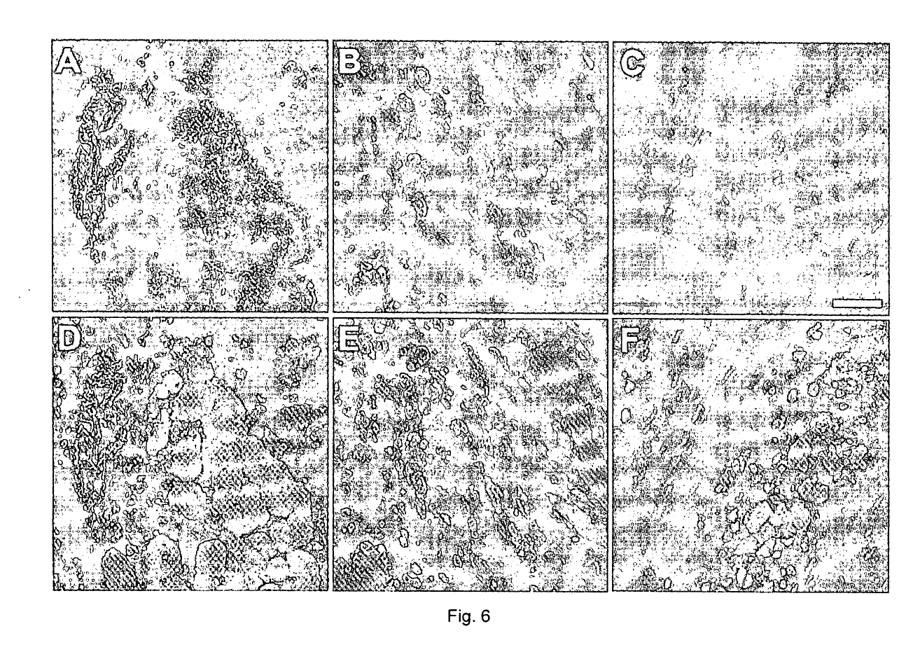

GATA4 responsive gene expression: We investigated the effect of intercellular delivery of GATA4 on the expression of cardiac myosin. Double-labeled immunohistochemical staining was used to identify the original area of transplanted cells within the infarct zone. The VP22 chimeric proteins have myc tags in the C-terminals. Using myc staining we traced injected cells in the peri-infarct zone 6 weeks after cell transplantation. In the peri-infarct zone around the needle track from cell injection there were GATA4:VP22 and GFP:VP22 expressing cells (FIGS. 6A and 6B). In these areas, we found in the animals that received GATA4:VP22 expressing RCF a relatively high expression of cardiac myosin (CM) heavy chain (FIG. 6D) based on CM red fluorescence staining using constant laser power and exposure times, as well as hypertrophied cardiac myocytes compared to control groups GFP:VP22 (FIG. 6E) and GFP (FIG. 6F).

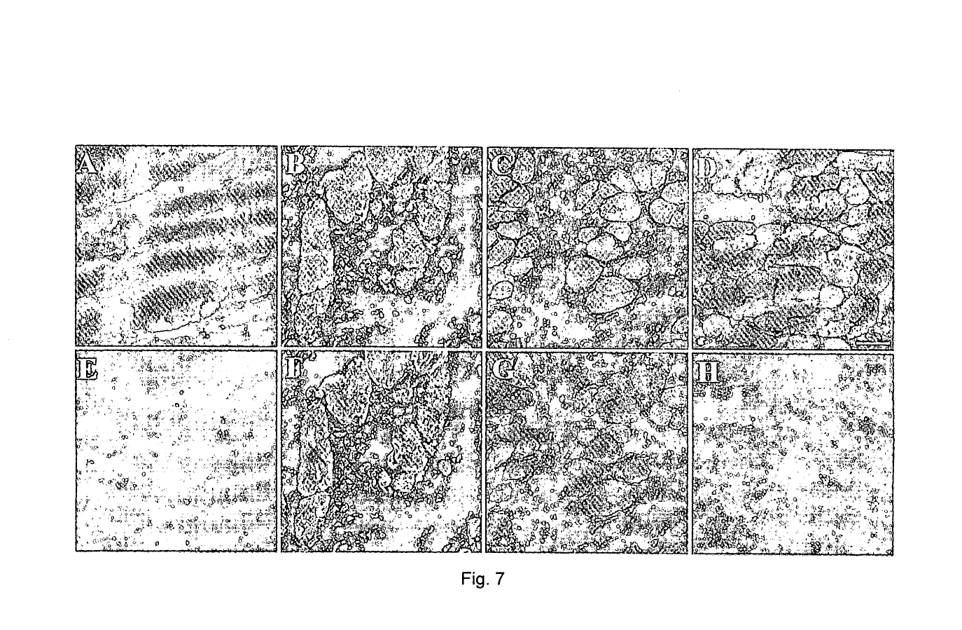

GATA4 has been shown to induce anti-apoptotic proteins in the heart following myocardial infarction. Recently, anti-apoptotic protein BCL2 was identified as a direct target of GATA4 and could mediate the prosurvival effects of GATA4. We similarly observed a significant increase in the level of BCL2 expression in the infarct border zone in animals that received GATA4:VP22 (FIG. 7B). No increase in BCL-2 expression was observed in areas remote from the infarct zone (FIG. 7D) or in control group of GFP:VP22 (FIG. 7C).

### Discussion

Our data provide the first study to investigate the role of transcription-factor delivery by a cell-penetrating peptide using a cell based gene transfer approach in ischemic cardiomyopathy. We show the non-secreted transcription factor GATA4 carried by cell penetrating peptide VP22 and combined with cell transplantation can be transported into the injured myocardium and function as trophic nuclear factor that modulates left ventricular remodeling. Our data demonstrates that the chronic over-expression of GATA4 in the infarct border zone had several effects ultimately leading to improvement in cardiac function. Furthermore, intercellular delivery of GATA4 induced myocyte hypertrophy (FIG. 5), which could temporarily protect heart to compensate for the condition of myocardial infarction. Moreover, we found that intercellular delivery of GATA4 induced a strong cardiac myosin overexpression and hypertrophy of cardiomyocytes around injection site in the peri-infarct zone compared with control (FIG. 6). Thus sustained reconstitution of transcription factor GATA4 in cardiogenesis and proper morphology around the peri-infarct zone may exert favorable effects on myocardium repair and preservation. All these findings are associated with enhanced LV function (FIG. 2) and improved ventricular morphology (FIG. 3). Consistent with the role of GATA4 in embryonic and injured adult heart, intercellular delivery of GATA4 in the infarcted adult heart may improve the LV function through multiple ‘combined’ monotherapies (i.e., cardiogenesis, antiapoptosis, morphogenesis) and using this new delivery system to deliver GATA4 in the infarcted myocardium may reduce the extent of left ventricular scarring and enhance preservation of left ventricular function after myocardial infarction.

To date, the role of zinc-finger transcription factor GATA4 has been mostly studied and characterized extensively during embryogenesis and the majority of these studies have focused on the role of GATA4 in the regulation of cardiac development and differentiation of myocyte in embryos. Since it controls a number of critical functions in the heart, including embryonic cardiogenesis and proper cardiac morphogenesis early in the heart development, most of studies were focused on GATA4 knockout mice with failed cardiogenesis and early lethality. However these studies did not examine the importance of GATA4 in regulating heart failure in the adult since transcription factor GATA4 can also regulate cardiac hypertrophic response and myocyte survival under the myocardial injury in the adult. Recent report showed that deletion of GATA4 specifically in heart using Cre-lox-dependent conditional gene targeting approach could observe a progressive and dosage-dependent deterioration in cardiac function and dilation in adulthood, and compromised in the ability to cardiac hypertrophy and myocyte viability. On the contrary to study the deterioration effect of cardiac-specific deletion of GATA4 in heart, in our present work it is the first time to observe the benefit effect on improvement of LV function when overexpressing GATA4 in the adult infarcted myocardium. Our data showed that sustained intercellular delivery of GATA4 in the injured adult heart causes activation of nuclear transcription factor GATA4 in myocardium repair (FIG. 2 and FIG. 3) culminating in improved clinical outcome.

In our study, improvement in LV function was mediated by an increase of radial strain in both peri-infarct and remote-infarct zone in the GATA4 treatment group compared with control (FIG. 2C). It appears somewhat unexpected that radial strains improvement was not confined to the treated (borderline) zones, but extended into the remote zones. The reason lies in that the strain not only reflects local properties of the myocardium, but may also be determined by the interaction of these local properties and regional stress. In end-systole, stress-strain relationships are linear, with the slope of this relationship reflecting regional contractility. After myocardial infarct, regional stress increases in both remote and infarct zones. Stress decrease by the removal of fibrosed myocardium improves regional fractional shortening (a measure very much related to radial strain) of remote regions. Therefore, a regional improvement of myocardial properties in any segment will lead to better global contraction, smaller end-systolic volume, and therefore reduction of stresses and improvement in strains in all of LV regions. The only regions in which improvement will not occur, of course, are regions with complete or near-complete fibrous replacement, with the strains virtually nil due to high tensile properties of fibrous tissue.

Moreover, this improved LV function was associated with higher survival rate in the GATA4 treatment group (FIG. 4). As we know, the limited mitotic capacity of adult cardiomyocyte restricts the repair of the ischemic myocardium leading to replacement by fibrotic tissue, which disrupts proper contractile function resulting in decreased cardiac performance. Cell-based intercellular delivery of GATA4 might prevent the post-infarct remodeling and death rate by direct protecting with anti-apoptotic effects in cardiomyocytes (FIG. 7) and preserving ventricular morphology by reducing fibronecrosis (FIG. 3), inducing cardiac myosin overexpression in cardiomyocyte (FIG. 6) and myocardium hypertrophy in the peri-infarct zone (FIG. 5). Since there was no function improvement in the infarct zone, GATA4 might only function in the survival and remodeling of spared myocardium but not dead myocardium. Cell penetrating peptides have been used to deliver a vast range of different biologically active compounds in different tissues. Our data provide the first evidence using cell-penetrating peptide to treat ischemic cardiomyopathy. As compared with other delivery vectors such that electroporation and microinjection are harsh and impractical to use in vivo, and liposome encapsulation and receptor-mediated endocytosis are limited by the lack of targeting and low yield of delivery, peptide-based delivery is to date the only method that succeeds in delivering a cargo without disturbing the plasma membrane and can be applicable in vivo. From the analysis of survival rate (FIG. 4), we provide evidence that cell-based intercellular delivery could provide sustained release of functional proteins in the injured heart without causing toxic side effects.

In conclusion, we developed a new carrier system, which combined cell transplantation with cell-penetrating peptide VP22 to induce sustained local intercellular delivery of functional transcription factors such as GATA4 in the injured heart. This system was feasible to induce remodeling of the injured heart late after myocardial infarction. Not surprisingly the sustained delivery of a transcription factor resulted in multiple changes in the myocardium including alterations in cardiac fibrosis, local hypertrophy in cardiac myocytes, increases in gene expression resulting in improved cardiac remodeling and cardiac function.

From the above description of the invention, those skilled in the art will perceive improvements, changes and modifications. Such improvements, changes and modifications within the skill of the art are intended to be covered by the appended claims.

Having described the invention, we claim:

1. A cell transfected to express a transcription factor-cell-penetrating peptide (TF-CPP) fusion protein.

2. The cell of claim 1, the TF including at least one DNA-binding motif selected from the group consisting of a helix-turn-helix motif, a zinc finger motif, a leucine zipper motif, a basic-helix-loop-helix motif, a G-quadruplex motif, and combinations thereof.

3. The cell of claim 2, the TF comprising a GATA-box binding protein.

4. The cell of claim 3, the GATA-box binding protein being selected from the group consisting of GATA1, GATA2, GATA3, GATA4, GATA5, GATA6, and combinations thereof.

5. The cell of claim 1, the CPP being selected from the group consisting of a HIV-1-trans-activating polypeptide, a Drosphila antennapedia homeodomain polypeptide, a herpes simplex-1 virus VP22 polypeptide, signal-sequence-based polypeptides, transportan, amphiphilic model polypeptides, homologs, fragments, variants and mutants thereof having translocational activity.

6. The cell of claim 1, the TF-CPP fusion protein comprising a GATA4-VP22 fusion protein.

7. The cell of claim 1, being a eukaryotic cell or a prokaryotic cell.

8. The cell of claim 1, being biocompatible with tissue of a subject to which the cell is administered for treatment of the tissue.

9. A pharmaceutical composition comprising a cell genetically engineered to express a GATA4-VP22 fusion protein.

10. The pharmaceutical composition of claim 9, the GATA4-VP22 fusion protein being capable of translocating across the plasma membrane of at least one cardiac cell.

11. The pharmaceutical composition of claim 9, the cell being biocompatible with tissue of a subject being treated.

12. The pharmaceutical composition of claim 9, the cell comprising an autologous cells or allogeneic cell of a subject being treated.

13. A method for treating cardiovascular disease, the method comprising:
preparing at least one cell delivery vehicle, the at least one cell delivery vehicle expressing a transcription factor-cell-penetrating peptide (TF-CPP) fusion protein; and
administering the at least one cell delivery vehicle to a cardiac target site, the cardiac target site comprising at least one cardiac cell.

14. The method of claim 13, delivery of the at least one cell delivery vehicle to the cardiovascular target site enabling the TF-CPP fusion protein to cross the plasma membrane of the at least one cardiac cell.

15. The method of claim 13, the TF including at least one DNA-binding motif selected from the group consisting of a helix-turn-helix motif, a zinc finger motif, a leucine zipper motif, a basic-helix-loop-helix motif, a G-quadruplex motif, and combinations thereof.

16. The method of claim 15, the TF comprising a GATA-box binding protein.

17. The method of claim 16, the GATA-box binding protein being selected from the group consisting of GATA1, GATA2, GATA3, GATA4, GATA5, GATA6, and combinations thereof.

18. The method of claim 13, the CPP being selected from the group consisting of a HIV-1-trans-activating polypeptide, a Drosphila antennapedia homeodomain polypeptide, a herpes simplex-1 virus VP22 polypeptide, signal-sequence-based polypeptides, transportan, amphiphilic model polypeptides, homologs, fragments, variants and mutants thereof having translocational activity.

19. The method of claim 13, the TF-CPP fusion protein comprising a GATA4-VP22 fusion protein.

20. The method of claim 13, the at least one cell delivery vehicle comprising an autologous or allogeneic cell that is biocompatible with the cardiac cells. eukaryotic cell or a prokaryotic cell.

21. The method of claim 13, the cardiovascular disease being selected from the group consisting of arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.
